(19) United States
(12) Patent Application Publication
(10) Pub. No.: US 2015/0196362 A1
(43) Pub. Date: Jul. 16, 2015

(54) Packable Animal First Aid Kit

(71) Applicant: Lynne Kunitz, Issaquah, WA (US)

(72) Inventor: Lynne Kunitz, Issaquah, WA (US)

(21) Appl. No.: 14/157,208

(22) Filed: Jan. 16, 2014

(51) Int. Cl. A61B 19/02

(52) U.S. Cl. CPC A61B 19/02; A61B 2019/0202

(57) ABSTRACT

The instant application discloses, among other things, an animal first aid kit. The animal first aid kit may comprise any container capable of holding its contents. The contents may include medicines and supplies that allow the user to evaluate an animal or to tend to it by providing comfort, controlling bleeding, or preventing further injury or infection, or example. The animal first aid kit may also include a pamphlet describing its contents and providing instructions on its use. In one embodiment, the pamphlet may direct the user to obtain a prescription for a particular device or medicine, for example, for an antihistamine such as Diphenhydramine.

### FIELD

This disclosure generally relates to a Packable Animal First Aid Kit.

### BACKGROUND

Animals may sustain a variety of injuries, accidents, and health conditions during their lifespan, with the majority occurring outside the home. Owners or caretakers of animals may benefit from having the proper medical supplies on hand so that they may promptly administer first aid until further help, if necessary, is obtained.

### SUMMARY

A Packable Animal First Aid Kit containing a variety of components that may provide a convenient way for a person to assist an injured or sick animal. Owners or caretakers may also call an animal emergency clinic to be directed to use the supplies available in the kit. It is not meant to replace proper veterinary care, but to allow first aid to be administered until proper care is obtained

The instant application discloses, among other things, a Packable Animal First Aid Kit, which may comprise a variety of components. The Packable Animal First Aid Kit may be configured to allow a user to treat any domesticated pet, or it may be tailored for a specific type of animal, for example, canines. The Packable Animal First Aid Kit may include medical supplies such as, but not limited to, triple antibiotic ointment, hydrogen peroxide tablets, electrolyte tablets, saline solution, bandages, gauze, dressings, styptic powder, cotton swabs, ear cleaner, gloves, scissors, and forceps. The Packable Animal First Aid Kit may also include an instructional pamphlet describing the contents. The pamphlet may include instructions directing a user to obtain a prescription for or purchase over-the-counter a particular medical device or medicine, for example, an antihistamine.

A Packable Animal First Aid Kit may be small and packable, and may be configured to be easy to carry when doing various activities, such as walking, hiking, camping, or boating.

A person having skill in the art will recognize that Packable Animal First Aid Kit may be comprised of various components. It may also be made of different materials and have different shapes, sizes, and colors.

### BRIEF DESCRIPTION OF THE DRAWINGS

FIG. 1 is a perspective view of Animal First Aid Kit, according to one embodiment.

FIG. 2 is an interior view of Animal First Aid Kit, according to one embodiment.

FIG. 3 is an example of Packable Animal First Aid Kit instructional booklet, according to one embodiment.

Like reference numerals are used to designate like parts in the accompanying drawings.

### DETAILED DESCRIPTION

A more particular description of certain embodiments of Packable Animal First Aid Kit may be had by references to the embodiments shown in the drawings that form a part of this specification, in which like numerals represent like objects.

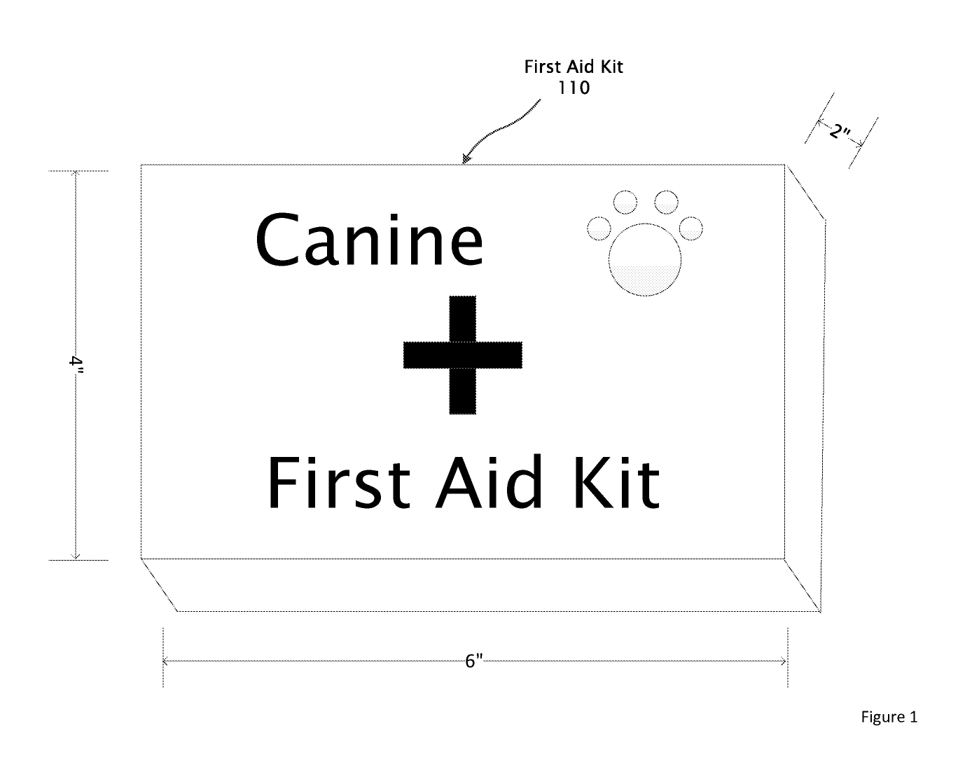

FIG. 1 is a perspective view of Animal First Aid Kit, according to one embodiment. Packable Animal First Aid Kit may comprise a container, such as a box, bag, or envelope, for example. The container may be made of any material including, but not limited to, plastic, nylon, cotton, metal, silicone, rubber, wood, or carbon fiber. In one embodiment, the Packable Animal First Aid Kit may be designed to withstand various environmental conditions such changes in temperature, humidity, altitude, or movement. The Packable Animal First Aid Kit may also be configured in ways that facilitate transportability and storage; for example, it may include handles, straps, or wheels, or it may have the ability to expand or contract in size.

In this example, a kit configured for use with dogs is displayed. This example is six inches long, four inches wide, and two inches deep. A smaller or larger container may be used. Small sizes, up to eight inches by six inches by three inches, may be more suitable for packing.

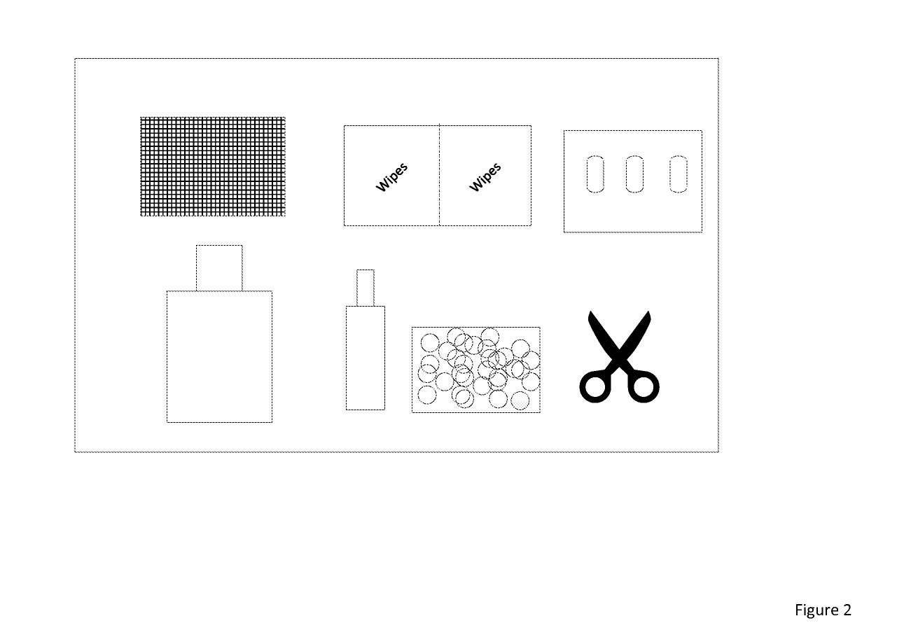

FIG. 2 is an interior view of Animal First Aid Kit, according to one embodiment. Packable Animal First Aid Kit may allow a user to treat any domesticated pet, or it may be configured for a specific type of animal, for example, dogs. A person skilled in the art will understand that Packable Animal First Aid Kit may include various types of medicines, supplies, and tools that may enable the user to tend to the animal by providing comfort, controlling bleeding, or preventing infection. Packable Animal First Aid Kit contents may include, but are not limited to, triple antibiotic ointment, hydrogen peroxide tablets, electrolyte tablets, saline solution, bandages, gauze, dressings, styptic powder, cotton swabs, ear cleaner, gloves, scissors, and forceps. In the example shown, Packable Animal First Aid Kit may contain gauze, antiseptic wipes, hydrogen peroxide tablets, saline solution, triple antibiotic ointment, cotton swabs, and scissors.

Packable Animal First Aid Kit may also include an instructional pamphlet, providing instructions on how to use one or more of the contents.

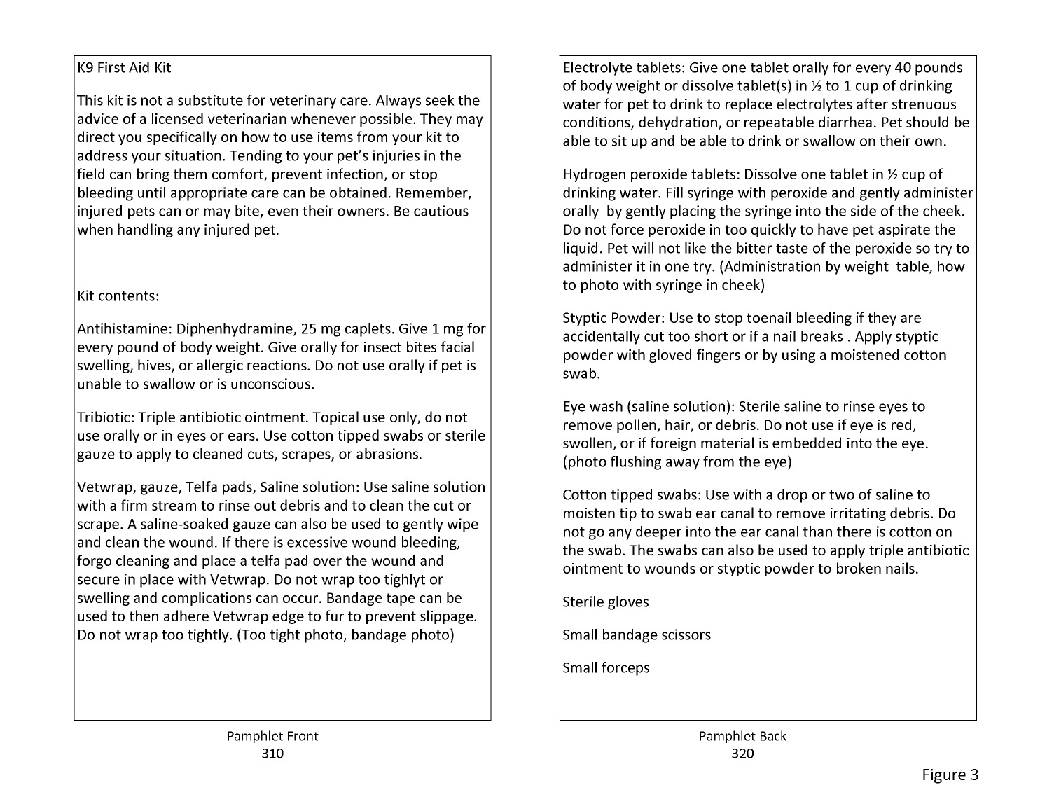

FIG. 3 is an example of a Packable Animal First Aid Kit instructional pamphlet 310, 320, according to one embodiment. The pamphlet may describe the contents of the Packable Animal First Aid Kit and provide instructions for its use. The pamphlet may also have direct the user to obtain a prescription from a licensed professional, such as a veterinarian, for a particular type of device or an over-the-counter medicine, for example, for an antihistamine. The pamphlet may also contain images, for example photos of proper ways to bandage or treat particular injuries. The pamphlet may be laminated and double sided, or it may be a booklet several pages long.

One having skill in the art will recognize that many different designs of Packable Animal First Aid Kit may fall within the spirit and scope of this disclosure, including, for example, varied contents and instructions.

While the detailed description above has been expressed in terms of specific examples, those skilled in the art will appreciate that many other configurations could be used. Accordingly, it will be appreciated that various equivalent modifications of the above-described embodiments may be made without departing from the spirit and scope of the invention.

The foregoing description of various embodiments of the invention has been presented for the purposes of illustration and description. It is not intended to be exhaustive or to limit the invention to the precise form disclosed. It is intended that the scope of the invention be limited not by this detailed description, but rather by the claims appended hereto. The above specification, examples and data provide a complete description of the manufacture and use of the invention. Since many embodiments of the invention can be made without departing from the spirit and scope of the invention, the invention resides in the claims hereinafter appended.

1. A packable animal first aid kit, comprising:
medicines and supplies for evaluating and treating a sick or injured animal;
a packable container configured to hold the medicines and supplies, the packable container having dimensions no larger than eight inches by six inches by three inches; and
an instructional pamphlet.

2. The packable animal first aid kit of claim 1, wherein the packable container is made of one or more materials selected from the group containing plastic, carbon fiber, nylon, cotton, metal, silicone, rubber, or wood.

3. The packable animal first aid kit of claim 1, further comprising an attachment means enabling the kit to be secured to a person, animal, or object.

4. The packable animal first aid kit of claim 1, further designed to withstand a change in temperature, humidity, altitude, or movement.

5. The packable animal first aid kit of claim 1, wherein the container is configured to expand or contract in size.

6. The packable animal first aid kit of claim 1, further comprising a prescription drug for a pet obtained from a veterinarian.

7. The packable animal first aid kit of claim 1, further comprising an over-the-counter drug for a pet obtained from a veterinarian.
